(19) United States
(12) Patent Application Publication
(10) Pub. No.: US 2015/0289878 A1
(43) Pub. Date: Oct. 15, 2015

(54) APPARATUS AND METHODS FOR APPLYING PRESSURE TO A FACE OF A SUBJECT

(71) Applicant: LACRIMA MEDICAL, LTD., Tel Aviv (IL)

(72) Inventors: Michael Gabriel Tal, Savyon (IL); Avinoam Gemer, Kiryat Ono (IL); Yael Kenan, Rosh Ha'ayin (IL)

(21) Appl. No.: 14/443,698

(22) PCT Filed: Nov. 20, 2013
(86) PCT No.: PCT/IL13/50959

§ 371 Date: May 19, 2015

Related U.S. Application Data
(60) Provisional application No. 61/728,749, filed on Nov. 20, 2012.

(51) Int. Cl. A61B 17/122; A61F 9/007

(52) U.S. Cl. CPC A61B 17/1227; A61F 9/00772

(57) ABSTRACT

Apparatus is provided, comprising (i) at least two cushions (28), each defining a respective skin-contacting surface (22), configured to be placed on a respective skin site (48) that is superficial to a lacrimal drainage vessel of the subject; (ii) a mount (30), coupled to the cushions, and configured (a) to be placed on a portion of a face of the subject that does not include the skin sites, and (b) to conform to a contour of the portion of the face; and (iii) an adhesive layer (26), disposed on the mount and not disposed on the skin-contacting surfaces, and configured (a) to adhere the mount to the portion of the face, and (b) when adhered to the portion of the face, to cause the mount to provide a pressing force that presses the skin-contacting surface of each cushion against the respective skin site thereof. Other embodiments are described.

### CROSS-REFERENCES TO RELATED APPLICATIONS

The present application claims priority from U.S. Provisional Patent Application 61/728,749 to Tal, entitled, “Punctal occlusion device and methods of use,” filed Nov. 20, 2012, and incorporated herein by reference.

### FIELD OF THE INVENTION

Some applications of the present invention relate in general to devices and methods for applying pressure to tissue in the vicinity of the eye of a subject. More specifically, some applications of the present invention relate to non-invasive lacrimal drainage occlusion devices and methods of use thereof. Some applications of the present invention relate to apparatus and methods for treating pain using a device that applies pressure to tissue in the vicinity of the eye of the subject. Some applications of the present invention relate to apparatus and methods for improving sleep using a device that applies pressure to tissue in the vicinity of the eye of the subject.

### BACKGROUND

Eye dryness is a condition affecting numerous patients around the world especially in modern world conditions. One of the causes for eye dryness is increased evaporation or drainage of the tears so the eyes are left with less natural moisture.

Lacrimal glands secrete lacrimal fluid, which flows into the space between the eyeball and eyelids. When the eyes blink, the lacrimal fluid is spread across the surface of the eye. Lacrimal fluid gathers and is drawn into the lacrimal punctum, then flows through the lacrimal canaliculi (i.e. ducts) at the inner corner of the eyelids entering the lacrimal sac, then on to the nasolacrimal duct, and finally into the nasal cavity.

Pressuring the lacrimal canaliculi or lacrimal sac, and/or adjusting (e.g., occluding) the opening of the lacrimal punctum is known to positively affect tear drainage. One common treatment is punctal occlusion, usually with dedicated plugs inserted into the punctum to block the canaliculus and/or adjust the flow traveling therethrough.

Non-invasive lacrimal occlusion means are taught in prior publications, including for preventing drainage of medicament away from the eye, such as in U.S. Pat. No. 8,147,467, U.S. Pat. No. 5,515,872, U.S. Pat. No. 5,522,837, and U.S. Pat. No. 5,832,930.

### SUMMARY OF THE INVENTION

There is a need for a durable and a reliable device for applying mild, consistent and/or controlled pressure, including, usability at normal sleep conditions. There is also a need for non-invasive eye-drainage occluders characterized as single-use, low-cost, easy and fast to handle and deploy, and/or being fittable to a range of face sizes, as well as optionally being sterile-packed.

According to some applications of the present invention there is provided an eye-drainage occluding device that is preferably disposable and/or intended for single use and/or configured for prolonged mild pressing of a portion of the lacrimal drainage apparatus and/or for recoverably occluding a punctum.

There is therefore provided, in accordance with an application of the present invention, apparatus for treating a subject, the apparatus including:

at least two cushions, each cushion defining a respective skin-contacting surface, configured to be placed on a respective skin site of the subject, each skin site of the subject being superficial to at least one lacrimal drainage vessel of the subject;

a mount, coupled to the at least two cushions, and configured (1) to be placed on a portion of a face of the subject that does not include the skin sites, and (2) to conform to a contour of the portion of the face of the subject; and

an adhesive layer, disposed on the mount and not disposed on the skin-contacting surfaces, and configured: (1) to adhere the mount to the portion of the face of the subject, and (2) when adhered to the portion of the face of the subject, to cause the mount to provide a pressing force that presses the skin-contacting surface of each of the at least two cushions against the respective skin site thereof.

In an application, a portion of the adhesive layer that is furthest from the skin-contacting surfaces is disposed at least 1 cm from the skin-contacting surfaces.

In an application:

the cushions are positioned to cause a superficially-directed force to be applied to at least a portion of the mount when the mount provides the pressing force, and

the apparatus is configured such that the adhesive layer inhibits movement, in response to the superficially-directed force, of the portion of the mount away from the face.

In an application, the mount is configured to provide the pressing force at a generally constant force value across a range of strain values of the elastically-deformable member, the range of strain values including at least between 1 percent strain and 8 percent strain.

In an application, each of the respective skin sites is disposed superficially to a respective lacrimal drainage vessel of the subject, and the apparatus is configured to treat dry eye of the subject by, when the mount is adhered to the portion of the face, pressing the skin-contacting surface of each of the at least two cushions against a respective one of the skin sites disposed superficially to the respective lacrimal drainage vessel.

In an application, each of the respective skin sites is disposed superficially to a respective selected anatomical site selected from the group consisting of: a medial palpebral ligament of the subject and a part of an orbicularis oculi muscle of the subject, and the apparatus is configured to improve sleep of the subject by, when the mount is adhered to the portion of the face, pressing the skin-contacting surface of each of the at least two cushions against a respective one of the skin sites disposed superficially to the respective anatomical site.

In an application, at least the skin-contacting surface of each cushion is water-resistant.

In an application, each of the cushions includes a nitinol spring, configured to alter a degree of compression thereof in response to body heat of the subject.

In an application, the mount is at least in part flexible.

In an application, the mount is at least in part malleable.

In an application, the mount is at least in part rigid.

In an application, the adhesive layer is configured to adhere the mount to the face of the subject independently of any coupling of the apparatus to the face of the subject provided by the pressing force.

In an application, the apparatus is configured such that the pressing force is insufficient to secure the apparatus to the face of the subject in the absence of the adhesive layer.

In an application, the apparatus further includes at least one adjustment mechanism, mechanically disposed between a respective skin-contacting surface and the mount, and configured to adjust a juxtaposition of the skin-contacting surface and the mount.

In an application, the adjustment mechanism is configured to automatically adjust the juxtaposition of the respective skin-contacting surface and the mount if the pressing force exceeds a threshold force.

There is further provided, in accordance with an application of the present invention, apparatus for use with a face of a subject, the apparatus including:

a plurality of skin-contacting surfaces including at least (1) a first skin-contacting surface configured to be placed in contact with a first skin site of the subject between a nasal bridge of the subject and a first eye of the subject, and (2) a second skin-contacting surface configured to be placed in contact with a second skin site of a subject between the nasal bridge of the subject and a second eye of the subject;

at least one elastically-deformable member:
coupled to at least one surface selected from the group consisting of: the first surface and the second surface,
configured (1) to be deformed, by application of a deforming force, into a first state in which a first distance exists between the first surface and the second surface, and (2) upon removal of the deforming force, to elastically return toward a second state in which a second distance exists between the first surface and the second surface, the second distance being smaller than the first distance, and
when the first surface is disposed on the first skin site and the second surface is disposed on the second skin site, to provide a pressing force that presses the first surface against the first skin site and the second surface against the second skin site; and

an adhesive layer, configured, by being adhered to the face of the subject, to couple the elastically-deformable member to the face of the subject such that the first surface is retained at the first skin site and the second surface is retained at the second skin site.

In an application, the adhesive layer is not disposed on the first skin-contacting surface or on the second skin-contacting surface.

In an application:

the first and second skin-contacting surfaces are positioned to cause a superficially-directed force to be applied to at least a portion of the elastically-deformable member, by providing the pressing force to the first and second skin sites, and

the apparatus is configured such that the adhesive layer inhibits movement, in response to the superficially-directed force, of the portion of the elastically-deformable member away from the face.

In an application, the plurality of skin-contacting surfaces are coupled to the adhesive layer at least via the elastically-deformable member.

In an application, the elastically-deformable member is configured to cause the skin-contacting surfaces to exert a constant pressure on the respective skin sites during a treatment of at least 5 hours.

In an application, the second state of the elastically-deformable member includes an unconstrained state, and the second distance is less than 10 mm.

In an application, the at least one elastically-deformable member includes at least one elastically-compressible cushion.

In an application, the apparatus further includes a face pad, the adhesive layer being disposed on the face pad and configured to adhere the face pad to skin of the face of the subject.

In an application, the face pad is coupled to the plurality of skin-contacting surfaces via the elastically-deformable member.

In an application, a portion of the adhesive layer that is furthest from the skin-contacting surfaces is disposed at least 1 cm from the skin-contacting surfaces.

In an application, the portion of the adhesive layer that is furthest from the skin-contacting surfaces is disposed at least 2 cm from the skin-contacting surfaces.

In an application, the face pad includes a nose pad, the adhesive layer being configured to adhere the nose pad to skin of a nose of the subject.

In an application, the nose pad is shapeable to a contour of the nose of the subject.

In an application, the adhesive layer is configured to adhere to at least one sidewall of the nose of the subject.

In an application, the face pad includes a forehead pad, the adhesive layer being configured to adhere the forehead pad to skin of the forehead of the subject.

In an application, the adhesive layer is configured to adhere the elastically-deformable member to the face of the subject independently of any coupling of the apparatus to the face of the subject provided by the pressing force.

In an application, the elastically-deformable member is configured such that the pressing force is insufficient to secure the apparatus to the face of the subject in the absence of the adhesive layer.

In an application, the apparatus further includes at least one adjustment mechanism, mechanically disposed between the adhesive layer and the skin-contacting surfaces, and configured to adjust a juxtaposition of the adhesive layer and the skin-contacting surfaces.

In an application, the adjustment mechanism includes a mechanism selected from the group consisting of: a screw and a ratchet.

In an application, the adjustment mechanism is configured to automatically adjust the juxtaposition of the adhesive layer and the skin-contacting surfaces if the pressing force exceeds a threshold force.

In an application, the first skin site includes a first skin site that is superficial to at least one anatomical site selected from the group consisting of: a medial palpebral ligament of the subject and a part of an orbicularis oculi muscle of the subject, and the first surface is configured to be placed in contact with the first skin site that is superficial to the selected anatomical site.

In an application, the apparatus is configured to pull tarsi of eyelids of the subject by providing the pressing force.

In an application, the elastically-deformable member is configured to improve sleep of the subject by providing the pressing force.

In an application, the elastically-deformable member is configured to provide a pressing force of less than 150 grams force to each of the skin-contacting surfaces, when a distance between the first and second skin-contacting surfaces is greater than the second distance and less than 40 mm.

In an application, the elastically-deformable member is configured to provide a pressing force of less than 100 grams force to each of the skin-contacting surfaces, when the distance between the first and second skin-contacting surfaces is greater than the second distance and less than 40 mm.

In an application, the elastically-deformable member is configured to provide a pressing force of less than 50 grams force to each of the skin-contacting surfaces, when the distance between the first and second skin-contacting surfaces is greater than the second distance and less than 40 mm.

In an application, the elastically-deformable member is configured to provide a pressing force of less than 20 grams force to each of the skin-contacting surfaces, when the distance between the first and second skin-contacting surfaces is greater than the second distance and less than 40 mm.

In an application, the first skin-contacting surface is configured, when pressed against the first tissue site by the elastically-deformable member, to contact between 1 mm̂2 and 144 mm̂2 of skin of the subject.

In an application, the elastically-deformable member is configured to treat pain of the subject by providing the pressing force.

In an application, the elastically-deformable member is configured to treat migraine of the subject by providing the pressing force.

In an application, the elastically-deformable member is configured to treat headache of the subject by providing the pressing force.

In an application, the first skin site includes a first skin site that is superficial to at least one anatomical site selected from the group consisting of: a lacrimal sac of the subject, a lacrimal groove of the subject, and a lacrimal canaliculus of the subject of the subject, and the first surface is configured to be placed in contact with the first skin site that is superficial to the selected anatomical site.

In an application, the elastically-deformable member is configured to inhibit drainage of at least the first eye of the subject, by providing the pressing force.

In an application, the elastically-deformable member includes at least one material selected from the group consisting of: metal, plastic, foam, rubber, and silicone.

In an application, the elastically-deformable member includes nitinol.

In an application, the elastically-deformable member is configured to provide the pressing force at a generally constant force value across a range of strain values of the elastically-deformable member, the range of strain values including at least between 1 percent strain and 8 percent strain.

In an application, the apparatus further includes a mount, coupled to the elastically-deformable member, the adhesive layer being disposed on the mount.

In an application, the mount encases the elastically-deformable member.

In an application, the mount is flexible.

In an application, the apparatus further includes a plurality of cushions, each of the cushions defining a respective one of the skin-contacting surfaces.

In an application, each of the cushions includes a nitinol spring, configured to alter a degree of compression thereof in response to body heat of the subject.

In an application, each cushion has a diameter of between 1 mm and 15 mm.

In an application, each cushion has a height of between 2 mm and 20 mm.

In an application, each cushion has a volume of between 2 mm̂3 and 4500 mm̂3, in the absence of any force applied thereto.

In an application, each of the plurality of cushions includes at least one material selected from the group consisting of: metal, foam, rubber, silicone, and plastic.

In an application, at least the skin-contacting surface of each of the plurality of cushions is water-resistant.

In an application, an area of the adhesive layer is at least 1 cm̂2.

In an application, the area of the adhesive layer is at least 2 cm̂2.

In an application, the area of the adhesive layer is at least 8 cm̂2.

In an application, the apparatus further includes a restraining element, couplable to the elastically-deformable member and configured to restrain the elastically-deformable member in the first state.

In an application:

the apparatus is configured, while the restraining element restrains the elastically-deformable member in the first state, to be coupled to the face of the subject such that (1) the adhesive layer is adhered to the face of the subject, (2) the first surface is in contact with the first skin site, and (3) the second surface is in contact with the second skin site, and

the elastically-deformable member is configured to be subsequently moved toward the second state by at least in part decoupling the restraining element from the elastically-deformable member.

There is further provided, in accordance with an application of the present invention, a method for use with a face of a subject, the method including:

providing apparatus including: (1) a plurality of skin-contacting surfaces including at least a first skin-contacting surface and a second skin-contacting surface, (2) an elastically-deformable member configured (i) to be deformed, by application of a deforming force, into a first state in which a first distance exists between the first surface and the second surface, and (ii) upon removal of the deforming force, to elastically return toward a second state in which a second distance exists between the first surface and the second surface, the second distance being smaller than the first distance, and (3) an adhesive layer;

coupling the elastically-deformable member to the face of the subject by adhering the adhesive layer to the face of the subject; and

placing (i) the first skin-contacting surface in contact with a first skin site of the subject between a nasal bridge of the subject and a first eye of the subject and (ii) the second skin-contacting surface in contact with a second skin site of the subject between the nasal bridge of the subject and a second eye of the subject, such that the elastically-deformable member provides a pressing force that presses the first surface against the first skin site and the second surface against the second skin site.

In an application, providing the plurality of skin-contacting surfaces, the adhesive layer, and the elastically-deformable member, includes providing the plurality of skin-contacting surfaces coupled to the adhesive layer at least via the elastically-deformable member.

In an application, the step of placing includes treating pain of the subject by placing (i) the first skin-contacting surface in contact with the first skin site, and (ii) the second skin-contacting surface in contact with the second skin site, such that the elastically-deformable member provides the pressing force.

In an application, providing the adhesive layer includes providing an adhesive layer that is not disposed on the first skin-contacting surface or on the second skin-contacting surface.

In an application, providing the elastically-deformable member includes providing an elastically-deformable member configured to elastically return to a second state in which the second distance is between 10 mm and 45 mm.

In an application, providing the elastically-deformable member includes providing an elastically-deformable member that includes at least one material selected from the group consisting of: metal, plastic, foam, rubber, and silicone.

In an application, providing the elastically-deformable member includes providing an elastically-deformable member that includes at least one elastically-compressible cushion.

In an application:

the step of placing includes positioning the first and second skin-contacting surfaces such that providing the pressing force applies a superficially-directed force to at least a portion of the elastically-deformable member, and

adhering the adhesive layer includes adhering the adhesive layer to the face such that the adhesive layer inhibits movement, in response to the superficially-directed force, of the portion of the elastically-deformable member away from the face.

In an application, placing the first skin-contacting surface in contact with the first skin site includes placing the first skin-contacting surface in contact with a first skin site that is superficial to at least one anatomical site selected from the group consisting of: a medial palpebral ligament of the subject and a part of an orbicularis oculi muscle of the subject.

In an application, the step of placing includes, using the pressing force, pulling tarsi of eyelids of the subject.

In an application, the step of placing includes improving sleep of the subject by placing (i) the first skin-contacting surface in contact with the first skin site, and (ii) the second skin-contacting surface in contact with the second skin site, such that the elastically-deformable member provides the pressing force.

In an application, the step of placing includes, by placing (i) the first skin-contacting surface in contact with the first skin site, and (ii) the second skin-contacting surface in contact with the second skin site, such that the elastically-deformable member provides the pressing force, treating a condition of the subject selected from the group consisting of: hypertension, heart failure, attention deficit hyperactivity disorder (ADHD), diabetes, stroke, and anxiety.

In an application, the step of placing includes placing (i) the first skin-contacting surface in contact with the first skin site, and (ii) the second skin-contacting surface in contact with the second skin site, such that the elastically-deformable member provides a pressing force of less than 150 grams force to each of the skin sites.

In an application, the step of placing includes placing (i) the first skin-contacting surface in contact with the first skin site, and (ii) the second skin-contacting surface in contact with the second skin site, such that the elastically-deformable member provides a pressing force of less than 100 grams force to each of the skin sites.

In an application, the step of placing includes placing (i) the first skin-contacting surface in contact with the first skin site, and (ii) the second skin-contacting surface in contact with the second skin site, such that the elastically-deformable member provides a pressing force of less than 50 grams force to each of the skin sites.

In an application, the step of placing includes placing (i) the first skin-contacting surface in contact with the first skin site, and (ii) the second skin-contacting surface in contact with the second skin site, such that the elastically-deformable member provides a pressing force of less than 20 grams force to each of the skin sites.

In an application, the step of placing includes placing (i) the first skin-contacting surface in contact with the first skin site, and (ii) the second skin-contacting surface in contact with the second skin site, such that the first skin-contacting surface contacts between 1 mm̂2 and 144 mm̂2 of skin of the subject.

In an application, placing the first skin-contacting surface in contact with the first skin site includes placing the first skin-contacting surface in contact with a first skin site that is superficial to at least one anatomical site selected from the group consisting of: a lacrimal sac of the subject, a lacrimal groove of the subject and a lacrimal canaliculus of the subject of the subject.

In an application, the step of placing includes inhibiting drainage of at least the first eye of the subject by placing (i) the first skin-contacting surface in contact with the first skin site, and (ii) the second skin-contacting surface in contact with the second skin site, such that the elastically-deformable member provides the pressing force.

In an application, inhibiting draining includes inhibiting drainage of tears.

In an application, the method further includes applying eye drops to at least the first eye of the subject, and inhibiting drainage includes inhibiting drainage of the eye drops away from at least the first eye of the subject.

In an application, applying the eye drops includes applying eye drops that include a medication for treating a disease of the subject.

In an application, applying the eye drops includes applying eye drops that include a medication for treating glaucoma.

In an application:

the step of providing includes providing a face pad, coupled to the plurality of skin-contacting surfaces via the elastically-deformable layer, the adhesive layer being disposed on the face pad, and

the step of adhering includes adhering the elastically-deformable member to the face of the subject by adhering the face pad to the face of the subject by adhering the adhesive layer to the face of the subject.

In an application, the step of adhering includes adhering, to the face of the subject, a portion of the adhesive layer that is furthest from the skin-contacting surfaces such that the portion is at least 1 cm from the skin-contacting surfaces.

In an application, the step of adhering includes adhering, to the face of the subject, the portion of the adhesive layer that is furthest from the skin-contacting surfaces such that the portion is at least 2 cm from the skin-contacting surfaces.

In an application, providing the face pad includes providing a nose pad, and the step of coupling includes coupling the elastically-deformable member to the face of the subject by adhering the nose pad to a nose of the subject by adhering the adhesive layer to the nose of the subject.

In an application, the method further includes shaping the nose pad to a contour of the nose of the subject.

In an application, adhering includes adhering the adhesive layer to at least one sidewall of the nose of the subject.

In an application, providing the face pad includes providing a forehead pad, and the step of coupling includes coupling the elastically-deformable member to the face of the subject by adhering the forehead pad to a forehead of the subject by adhering the adhesive layer to the forehead of the subject.

In an application, the method further includes shaping the nose pad to a contour of the forehead of the subject.

In an application, the method further includes decoupling the elastically-deformable member from the face of the subject at least 5 hours after the step of coupling.

In an application, decoupling includes decoupling the elastically-deformable member from the face of the subject at least 8 hours after the step of coupling.

In an application, the placing step includes, subsequent to placing the first skin-contacting surface in contact with the first skin site and the second skin-contacting surface in contact with the second skin site, moving the elastically-deformable member toward the second state thereof by releasing a restraining force.

In an application, the method further includes, prior to the placing step, deforming the elastically-deformable member into the first state.

In an application, the apparatus includes a restraining member that restrains the elastically-deformable member in the first state thereof, and releasing the force includes decoupling the restraining member from the elastically-deformable member.

In an application, providing the apparatus includes providing apparatus that includes a mount, coupled to the elastically-deformable member, the adhesive layer being disposed on the mount.

In an application, providing the mount includes providing a mount that encases the elastically-deformable member.

In an application, providing the mount includes providing a mount that is flexible.

In an application, providing the mount includes providing a mount that is at least in part malleable.

In an application, providing the mount includes providing a mount that is at least in part rigid.

In an application, providing the apparatus includes providing apparatus that includes a plurality of cushions, each of the cushions defining a respective one of the skin-contacting surfaces.

In an application, providing the plurality of cushions includes providing a plurality of cushions, each of the cushions having a diameter of between 1 mm and 15 mm.

In an application, providing the plurality of cushions includes providing a plurality of cushions, each of the cushions having a height of between 2 mm and 20 mm.

In an application, providing the plurality of cushions includes providing a plurality of cushions, each of the cushions having a volume of between 2 mm̂3 and 4500 mm̂3, in the absence of any force applied thereto.

In an application, providing the plurality of cushions includes providing a plurality of cushions that each include at least one material selected from the group consisting of: metal, foam, rubber, and silicone.

In an application, providing the plurality of cushions includes providing a plurality of cushions, the skin-contacting surface of each cushion being water-resistant.

In an application, providing the adhesive layer includes providing an adhesive layer that is configured to adhere the elastically-deformable member to the face of the subject independently of any coupling of the apparatus to the face of the subject provided by the pressing force.

In an application, providing the elastically-deformable member includes providing an elastically-deformable member that is configured such that the pressing force is insufficient to secure the apparatus on the face of the subject in the absence of the adhesive layer.

There is further provided, in accordance with an application of the present invention, a lacrimal drainage occlusion device including:

a malleable strip portion and/or wire sized and shaped to selectively surround, at least partly, a specific human face portion including an anatomical structure selected from the group consisting of: a lacrimal canaliculus and a lacrimal sac; and

an adhesive layer provided on an inner side of the strip portion, configured to adhere the strip portion to said human face portion, and said device, when left deployed on said human face portion, self-compresses and particularly presses the selected anatomical structure.

In an application, the device further includes a disposable covering provided over the adhesive layer and being removable prior to device deployment.

In an application, the device further includes at least one protruding portion outwardly protruding away from the inner side, configured to press onto the selected anatomical structure when said device is deployed on said human face portion.

In an application, the device further includes removable strip straightening means, configured to maintain said strip portion and/or wire straight, being removable prior or during device deployment, thereby allowing self-compression thereof.

In an application, said wire is formed of Ni—Ti alloy being slightly above transformation temperature when in contact with the face portion.

In an application, the device further includes at least one member fixedly extending along a length of the strip portion, configured to compress the strip portion such that two end portions thereof meet at a nominal distance substantially smaller than a width of said human face portion.

In an application, said at least one member includes an elastic metal member bendable to a singular non-stressed shape.

There is further provided, in accordance with an application of the present invention, a method of treating dry eyes, the method including:

providing a lacrimal drainage occlusion device including a malleable nasal strip and/or wire portion sized and shaped to selectively surround, at least partly, a portion of a human nose; and

applying device to at least a portion of a human nose such that the device surrounds, at least partly, a portion of the human nose with a first portion of the device positioned to apply pressure to a right lacrimal canaliculus and a second portion of the device positioned to apply pressure to a left lacrimal canaliculus;

and the remains positioned on at least a portion of the human nose, applying pressure to the right and left lacrimal canaliculi, for a prolonged duration of time.

In an application, the method further includes removing a straightening element from the device, and upon removing the straightening element, the malleable nasal strip and/or wire portion compresses into a curved form.

In an application, the device remains positioned on at least a portion of the human nose for more than 5 minutes.

In an application, the device remains positioned on at least a portion of the human nose for more than 2 hours.

In an application, the device remains positioned on at least a portion of the human nose overnight.

The present invention will be more fully understood from the following detailed description of applications thereof, taken together with the drawings, in which:

### BRIEF DESCRIPTION OF THE DRAWINGS

FIGS. 1A-C and 2A-C are schematic illustrations of a device for treating a subject, in accordance with some applications of the invention;

FIG. 3 is a schematic illustration of a device for treating a subject, in accordance with some applications of the invention;

FIG. 4 is a schematic illustration of a device for treating a subject, in accordance with some applications of the invention;

FIGS. 5A-C are schematic illustrations of adjustment mechanisms for adjusting a distance between a central core portion and skin-contacting surfaces of a device for treating a subject, in accordance with some applications of the invention;

FIGS. 6-7 are schematic illustrations of adjustment mechanisms for adjusting a distance between skin-contacting surfaces and a longitudinal member and/or mount of a device for treating a subject, in accordance with some applications of the invention; and

FIGS. 8, 9A-C, and 10A-B are schematic illustrations of devices for treating a subject, in accordance with some applications of the invention.

### DETAILED DESCRIPTION OF EMBODIMENTS

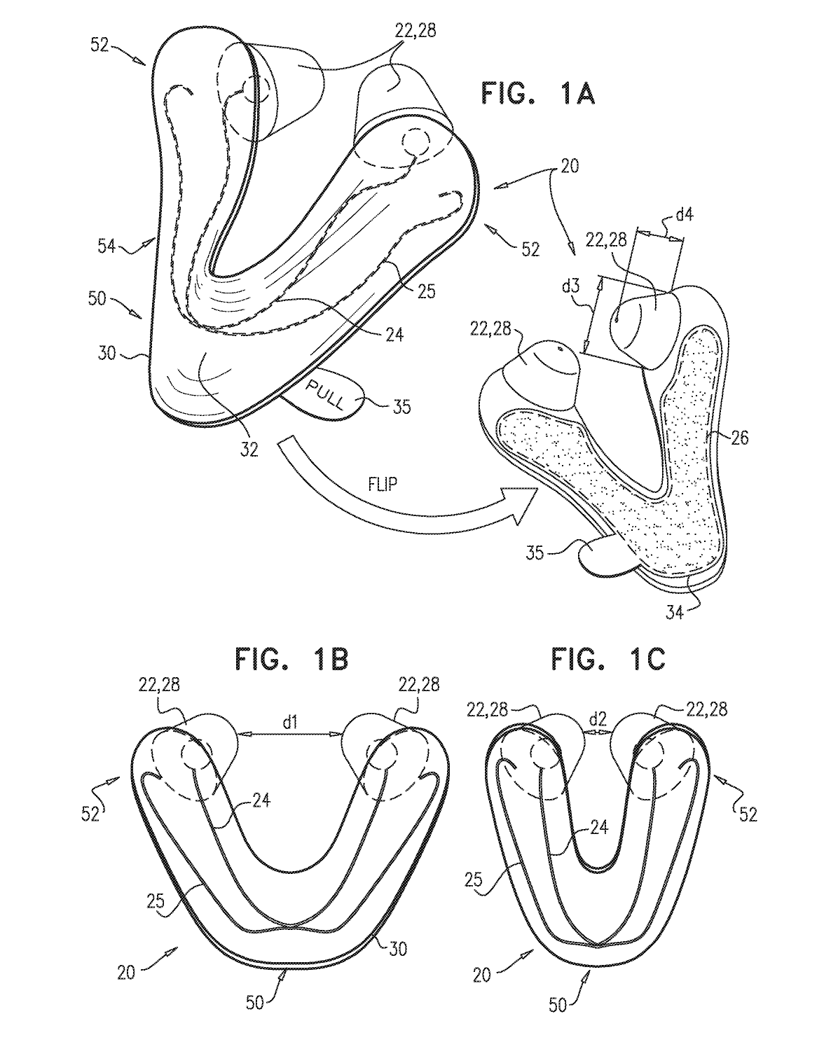

Reference is made to FIGS. 1A-C and 2A-C, which are schematic illustrations of a device 20 for treating a subject, in accordance with some applications of the invention. Device 20 has a plurality of skin-contacting surfaces 22, and comprises at least one elastically-deformable member, such as a longitudinal member 24, and an adhesive layer 26. Typically, device 20 comprises a plurality of cushions 28 that define skin-contacting surfaces 22 (e.g., an outer surface of each cushion defines a respective skin-contacting surface). Typically, device 20 comprises a mount 30, coupled to or being the same as longitudinal member 24, and adhesive layer 26 is disposed on the mount. Further typically, at least part of longitudinal member 24 is disposed within mount 30 (e.g., mount 30 encases member 24). Still further typically, mount 30 comprises a face pad, such as a nose pad 32, on which adhesive layer 26 is disposed. For example, a portion of mount 30 may serve as the face pad. For some applications, the face pad comprises a forehead pad, configured to adhere to the forehead of the subject, and typically shapeable to a contour of the forehead of the subject. For some applications, the face pad comprises both nose pad 32 and a forehead pad.

Longitudinal member 24 typically comprises an elastically-deformable material such as a metal (e.g., nitinol or stainless steel), a plastic, a rubber, silicone, and/or a foam. Longitudinal member 24 typically is shaped as a wire or a strip. Mount 30 typically comprises a flexible material (such as a fabric, a polymer, silicone, and/or a foam), suitable for contact with the skin for many hours.

Longitudinal member 24 is coupled to at least one (typically both) skin-contacting surfaces 22 (e.g., by being coupled to cushions 28), and is configured (1) to be deformed, by application of a deforming force, into a first state in which a first distance d1 exists between the first surface and the second surface (FIG. 1B), and (2) upon removal of the deforming force, to elastically return toward a second state in which a second distance d2, smaller than the first distance, exists between the first surface and the second surface (FIG. 1C). That is, the longitudinal member provides device 20 with elastic deformability such that the device is deformable into a more open state by applying a deforming force, and automatically returns to a more closed state when the deforming force is removed.

Typically, distance d2 is less than 20 mm, such as less than 15 mm, e.g., less than 10 mm. For some applications, distance d2 is generally zero (e.g., surfaces 22 touch when longitudinal member 24 is in the second state).

For some applications, distance d1 is greater than 25 mm, less than 45 mm, and/or between 25 mm and 45 mm, e.g., between 30 and 35 mm, such as about 33 mm.

Device 20 is configured to be placed on a face 40 of a subject, such that (1) each skin-contacting surface 22 is placed in contact with a respective skin site 48 of the subject between a nasal bridge 42 of the subject and a respective eye 44 of the subject, and (2) adhesive layer 26 of nose pad 32 is adhered to a nose 46 of the subject, thereby holding device 20 in place (see FIGS. 2A-C). It is to be noted that surfaces 22 are coupled to adhesive layer 26 at least via elastically-deformable member 24. For some applications, surfaces 22 are coupled to adhesive layer 26 also via mount 30.

For some applications, adhesive layer 26 comprises a tacky silicone gel. It is to be noted that any suitable biocompatible adhesive may be used. Typically, device 20 is provided with a release liner 34 (FIG. 1A) disposed over at least adhesive layer 26.

Once applied to the face, longitudinal member 24 provides (e.g., transfers) a pressing force that presses skin-contacting surfaces 22 against the respective skin sites. Furthermore, in this state, skin-contacting surfaces 22 (e.g., surfaces of cushions 28) typically cause a superficially-directed force to be applied to at least a portion of the elastically-deformable member (e.g., a portion on which adhesive layer 26 is disposed), by providing the pressing force to the respective skin sites. That is, the pressing of surfaces 22 into the skin sites typically results in a force that, in the absence of adhesive layer 26, would move the portion of the elastically-deformable member away from skin of the nose of the subject. Adhesive layer 26 inhibits such movement.

For some applications, device 20 is placed on a subject who suffers from dry eye, and is configured to treat dry eye by temporarily at least partially inhibiting drainage of tears from the eye via the lacrimal system. For example, skin sites 48 may be superficial to one or more respective structures of a lacrimal drainage system 10 of the subject (e.g., respective lacrimal canaliculi 12, lacrimal sacs 14, and/or lacrimal grooves of the subject), and device 20 may be configured to apply pressure to the structures such that the structures are at least partially occluded (see FIG. 2B).

For some applications, device 20 is placed on a subject prior to the application of eye drops, and the inhibition of drainage described hereinabove inhibits drainage of the eye drops, thereby increasing the efficacy thereof. For some applications, the eye drops comprise a medication for treating a disease of the subject, such as glaucoma.

For some applications, device 20 is placed on a subject who suffers from suboptimal sleep, and is configured to improve sleep. For example, skin sites 48 may be superficial to respective medial palpebral ligaments, and/or parts of (e.g., origins of) respective orbicularis oculi muscles of the subject, and device 20 may be configured to apply pressure to these ligaments and/or muscles. For some such applications, device 20 is configured such that such pressing pulls tarsi of respective eyelids of the subject.

For some applications, device 20 is placed on a subject who suffers from pain, such as migraine, and is configured to treat the pain. For example, skin sites 48 may be superficial to a nasal bone of the subject, and device 20 may be configured to apply pressure to these sites. It is hypothesized that, for some applications, pressure (e.g., light pressure) on such sites treats the pain by desensitizing the subject. For some applications, continuous pressure is used (e.g., as described elsewhere herein, mutatis mutandis). For some applications, a desensitization technique that provides frequent short periods of pressure is used.

Typically, device 20 (e.g., the elastically-deformable member thereof, e.g., longitudinal member 24, and/or mount 30) is configured to provide a pressing force of less than 150 g force (e.g., less than 100 g force, e.g., less than 50 g force, such as less than 20 g force) to each skin-contacting surface 22 (and thereby to each skin site 48). Typically, device 20 is configured in this manner by being configured to apply such a pressing force when the distance between skin-contacting surfaces 22 is greater than that when the device is in the second state (e.g., when the distance is between that when the device is in the second “more closed” state, and that when the device is in the first “more open” state), e.g., while skin sites 48 are disposed between the skin-contacting surfaces and hold device 20 at least partly open. For example, for a subject in which skin sites 48 are disposed a given distance apart (e.g., 35 mm apart), device 20 is configured to apply such a pressing force when skin-contacting surfaces 22 are separated by the given distance (e.g., by 35 mm). Typically, device 20 is configured to apply such a pressing force when the distance between skin-contacting surfaces 22 is greater than second distance d2, and less than 40 mm.

It is to be understood that device 20 may be configured to apply such a pressing force to tissue sites of subjects having different distances between their tissue sites. For example, device 20 may be configured to apply such a pressing force when the distance between surfaces 22 is at any distance within a range of 20 mm-45 mm, at any distance within a sub-range of that range (e.g., 27-39 mm, 30-36 mm, 27-35 mm and/or 35-45 mm), or at a specific distance within that range. When surfaces 22 are pressed against respective skin sites 48, each of the surfaces typically contacts at least 1 mm̂2 and/or less than 144 mm̂2 (e.g., between 1 mm̂2 and 144 mm̂2, such as between 9 mm̂2 and 144 mm̂2) of skin of the subject.

Typically, device 20 (e.g., mount 30 thereof) generally defines a V-shape, having a central core region 50, and extremity regions 52 (e.g., “wings”) at which surfaces 22 (e.g., cushions 28) are disposed.

Nose pad 32 is at least partly shapeable to a contour of the nose of the subject, e.g., so as to facilitate contact, and thereby adhesion, between the skin of the nose and adhesive layer 26. Typically, nose pad 32 is shapeable, and adhesive layer 26 is disposed thereon, such that the adhesive layer is adherable to at least one sidewall 47 (e.g., both sidewalls) of the nose of the subject.

Typically, adhesive layer 26 has a surface area of at least 1 cm̂2 (e.g., at least 2 cm̂2, such as at least 8 cm̂2) and/or less than 100 cm̂2. For some applications, adhesive layer 26 comprises a plurality of adhesive layers, disposed on different regions of mount 30, and having a combined surface area of at least 1 cm̂2 (e.g., at least 2 cm̂2), and/or less than 100 cm̂2 (e.g., less than 50 cm̂2). As described hereinabove, adhesive layer 26 is typically disposed on mount 30. For some applications, adhesive layer 26 is not disposed on skin-contacting surfaces 22 (e.g., the adhesive layer is not disposed on cushions 28). For some applications, a portion of adhesive layer 26 that is furthest from the skin-contacting surfaces is disposed at least 1 mm (e.g., at least 5 mm, e.g., at least 1 cm, such as at least 2 cm) from skin-contacting surfaces 22. For example, adhesive layer 26 may be disposed (e.g., solely disposed) on regions of mount 30 that are configured to be placed against a sidewall 47 or a tip 49 of the nose of the subject, such as on core region 50 of the mount.

For some applications, adhesive layer 26 is adhered to face 40 of the subject prior to placement of surfaces 22 against skin sites 48 (e.g., while holding device 20 in the first, “open” state). For some applications, these steps are performed in reverse order. For some applications, placing adhesive layer 26 against the face deforms device 20 into the first “open” state. For some applications, surfaces 22 and adhesive layer 26 are placed against the face simultaneously.

Typically, release liner 34 is configured to be removed prior to application of the device to the face. Alternatively, release liner 34 may be configured such that at least part of the release liner is removable after the device is applied to the face. For example, surfaces 22 may be placed in contact with skin sites 48 and/or mount 30 may be at least partly positioned before release liner 34 is removed and adhesive layer 26 is adhered to the nose of the subject. For some applications, release liner 34 is placed in contact with the nose of the subject before removal of the release liner, therefore being sandwiched between adhesive layer 26 and the face (e.g., nose) of the subject, and is removed (e.g., pulled out) from between the adhesive layer and the face in order to adhere adhesive layer 26 to the face. For some applications, liner 34 is coupled to or defines a tab 35 that facilitates such removal.

The pressing force provided by longitudinal member 24 to both surfaces 22 (and thereby to both skin sites 48) typically clamps, between surfaces 22, a portion of the face of the subject (e.g., a portion posterior to bridge 42 of the nose of the subject, see FIG. 2C). Typically, adhesive layer 26 is configured to adhere device 20 (e.g., longitudinal member 24 thereof) to the face of the subject independently of any coupling provided by the pressing force (e.g., independently of any coupling provided by the clamping caused by the pressing force). For some applications, the clamping caused by the pressing force is insufficient to secure (e.g., to reliably secure) the apparatus to the face of the subject in the absence of the adhesive layer.

For some applications, device 20 is left in place for a duration of at least 15 min, such as at least 1 hour. For some applications, e.g., for treating dry eye, device 20 is left in place for a duration of at least 5 hours (e.g., at least 8 hours and/or overnight while the subject sleeps), such that the device exerts a generally constant pressure on skin sites 48 for the duration.

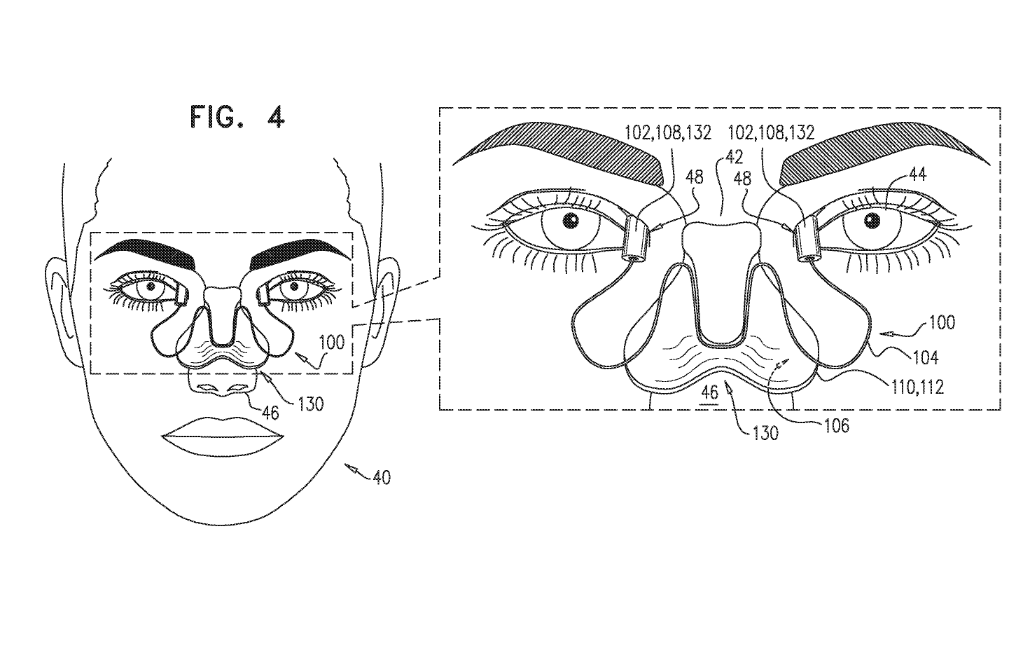

Typically, cushions 28 are elastically-compressible (i.e., comprise elastically-deformable members), and comprise a foam. Cushions 28 may alternatively or additionally comprise a rubber, silicone, a plastic, or a metal (e.g., an elastic wire). For some applications, and as shown in FIGS. 5A-C, cushions 28 comprise a spring 154, which provides at least some of the elastic compressibility to the cushion. Typically, a degree of compression of spring 154 at least in part defines a distance between surface 22 and longitudinal member 24. Spring 154 may comprise a metal, such as nitinol or stainless steel. Typically, one end of spring 154 is coupled to longitudinal member 24, and the other end of the spring is coupled to a pad 156 that presses surface 22 against skin site 48. For some applications, pad 156 may itself define skin-contacting surface 22.

Typically, each cushion 28 has a diameter d3 of at least 1 mm and/or less than 15 mm (e.g., between 1 and 15 mm, such as between 3 and 15 mm), a height d4 (i.e., away from mount 30) of at least 2 mm and/or less than 20 mm (e.g., between 2 and 20 mm, e.g., between 2 and 8 mm, e.g., between 3 and 7 mm, such as between 4 and 6 mm), and/or a volume of at least 2 mm̂3 and/or less than 4500 mm̂3 (e.g., between 2 mm̂3 and 300 mm̂3, or between 300 mm̂3 and 4500 mm̂3).

Although cushions 28 are shown as being generally round, and d2 is referred to as the diameter, it is to be noted that cushions 28 may have other shapes, and d2 may represent a width of the cushion, or a portion thereof.

For some applications, cushions 28 are water-resistant, e.g., so as to inhibit absorption of tears. For example, cushions 28 may have a water-resistant surface (e.g., skin-contacting surface 22 may be a water-resistant surface).

When device 20 is disposed on face 40 of the subject, when viewed from the front of the subject (FIG. 2A), an angle a1 is defined between (1) an axis x1 between (i) core region 50 and (ii) surface 22 and/or cushion 28, and (2) a central sagittal plane x2 of the subject (e.g., of the nose of the subject). Typically, angle a1 is greater than 15 degrees and/or less than 45 degrees (e.g., between 15 and 45 degrees, e.g., between 20 and 40 degrees, such as about 30 degrees).

When viewed from the side of the subject (FIG. 2B), an angle a2 is defined between (1) an axis x3 between (i) core region 50 and (ii) surface 22 and/or cushion 28, and (2) a transverse plane x4 of the subject. Typically, angle a2 is greater than 25 degrees and/or less than 45 degrees (e.g., between 25 and 45 degrees, e.g., between 30 and 40 degrees, such as about 36 degrees).

When viewed from above (FIG. 2C), an angle a3 is defined between (1) a direction x5 in which the pressing force presses each surface 22 against its respective skin site, and (2) an axis a6 between surfaces 22. Typically, angle a3 is greater than 30 degrees and/or less than 90 degrees, (e.g., between 30 and 90 degrees, e.g., between 45 degrees and 75 degrees, such as about 60 degrees). The adhesion of adhesive layer 26 to the nose of the subject typically facilitates such values of angle a3, e.g., by adhering device 20 to the face of the subject independently of any coupling (e.g., clamping) provided by the pressing force, as described hereinabove.

For some applications, portions of longitudinal member 24 and/or mount 30 are more rigid than other portions thereof. For example, for some applications, sidewall-contacting regions 54 (see FIG. 1A), disposed between central core region 50 and extremity regions 52, may be more rigid than the central core region and/or the extremity region. For some such applications, only central core region 50 is flexible, while regions 54 and 52 are rigid.

For some applications, in addition to or alternatively to the elastic deformability of longitudinal member 24, at least a portion of longitudinal member 24 is malleable.

For some applications, device 20 comprises a second longitudinal member 25, which is typically elastically-deformable and further typically is shaped as a wire or a strip. Typically, at least part of member 25 is disposed within mount 30 (e.g., mount 30 encases member 25). Longitudinal member 24 typically forms a V-shape, with a middle portion disposed at region 50 of mount 30, and end portions disposed at regions 52 of the mount, typically attached to cushions 28. Second longitudinal member 25 also typically forms a V-shape, with a middle portion disposed at region 50 of mount 30, and end portions disposed at regions 52 of the mount, but the end portions of member 25 are typically not attached to cushions 28. Second longitudinal member 25 typically pushes regions 52 of mount 30 against the skin of the subject, e.g., to facilitate adhesion, to provide structural integrity, and/or to provide stabilization.

Although longitudinal member 25 is referred to as a “second longitudinal member”, device 20 may comprise more than one such “second longitudinal member”, having the characteristics described herein for second longitudinal member 25. It is to be further noted that, although FIGS. 1A-C show device 20 comprising second longitudinal member 25, device 20 may not comprise member 25.

As described hereinabove, cushions 28 are typically elastically-compressible. For some applications, at least one of mount 30, longitudinal member 24, and/or second longitudinal member 25 is rigid and/or malleable, and cushions 28 comprise and/or function as the sole elastically-deformable members. For some such applications, device 20 does not comprise longitudinal member 24.

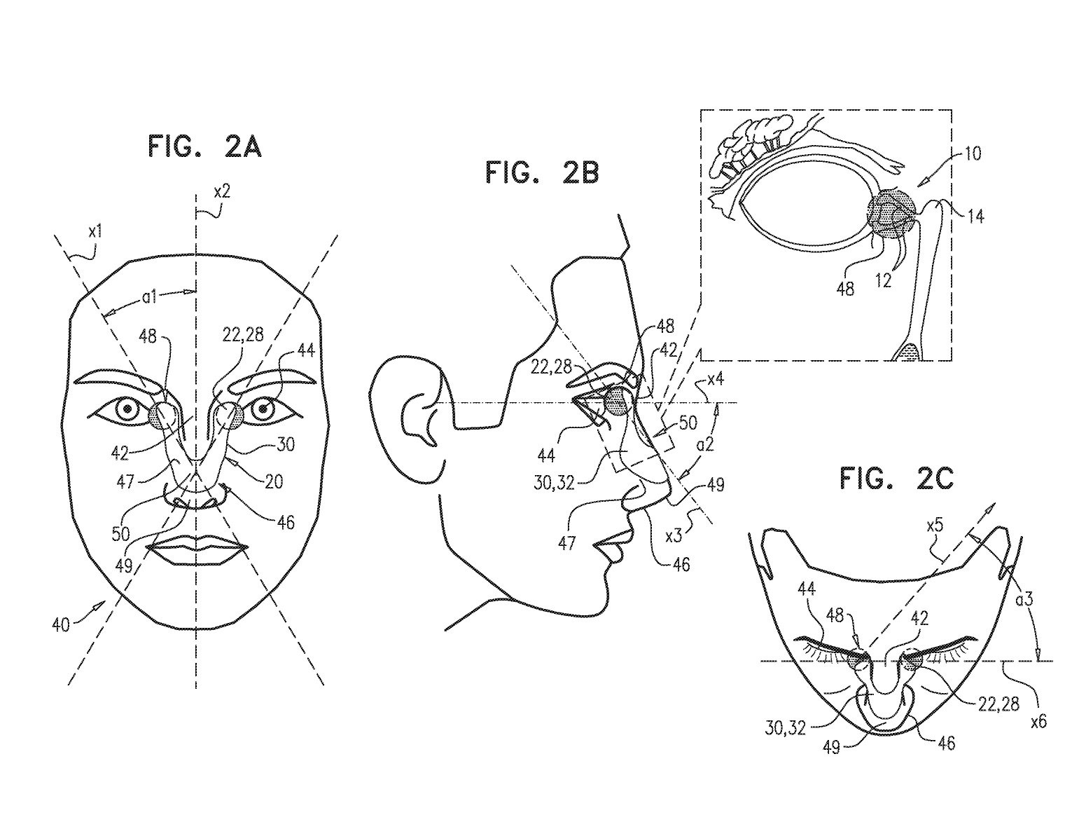

Reference is made to FIG. 3, which is a schematic illustration of a device 60 for treating a subject, in accordance with some applications of the invention. Device 60 has a plurality of skin-contacting surfaces 62, and comprises at least one elastically-deformable member, such as a longitudinal member 64, and an adhesive layer 66. Typically, device 60 comprises a plurality of cushions 68 that define skin-contacting surfaces 62. Typically, device 60 comprises a mount 70, coupled to or being the same as longitudinal member 64, and adhesive layer 66 is disposed on the mount. Further typically, at least part of longitudinal member 64 is disposed within mount 70 (e.g., mount 70 encases member 64). Alternatively, longitudinal member 64 may be attached to a surface of mount 70. Still further typically, mount 70 comprises a face pad, such as a nose pad 72, on which adhesive layer 66 is disposed. For example, a portion of mount 70 may serve as the face pad.

For some applications, components of device 60 are identical to, and/or function in the same manner as, identically-named components of device 20, described hereinabove, mutatis mutandis, except as described.

Device 60 (e.g., mount 70 thereof) is generally symmetrical, having a central core region 90, and a side region 91 on each side of core region 90. Side regions 91 are configured to be placed against respective sidewalls of the nose of the subject, and include or are coupled to respective extremity regions 92 at which surfaces 62 (e.g., cushions 68) are disposed.

Typically, device 60 comprises two longitudinal members 64, disposed at respective side regions 91 of the device. For some applications, device 60 further comprises a respective two second longitudinal members 65, disposed on respective sides of the device. Whereas each longitudinal member 64 is typically attached to a respective cushion 68, second longitudinal members 65 are typically not attached to cushions 68. For some applications, each longitudinal member 64 and respective second longitudinal member 65 are coupled to or are continuous with each other (e.g., are formed from a single wire or strip). For some applications, each member 64 is coupled to its respective member 65 at an end of the member 64 that is furthest from its respective cushion 68 (e.g., as shown in “option A” of FIG. 3). For some applications, each member 64 is coupled to its respective member 65 at an end of the member 64 that is closest to its respective cushion 68 (e.g., as shown in “option B” of FIG. 3).

For some applications, each side region 91 of device 60 is adhered to the nose of the subject (e.g., to a sidewall of the nose) independently of the adhesion of the other side region. Typically, device 60 is configured in this manner by each longitudinal member 64 being separate from the other. Typically, when each side region 91 is placed against the nose of the subject, its respective surface 92 (e.g., of cushion 68) is pressed against its respective skin site 48.

For some applications, portions of longitudinal member 64, longitudinal member 65, and/or mount 70 are more rigid than other portions thereof. For some applications, longitudinal member 64 and/or longitudinal member 65 is rigid along its entirety. For some applications, sidewall-contacting regions 94, disposed between central core region 90 and extremity regions 92, are more rigid than the central core region and/or the extremity region. For some such applications, only central core region 90 is flexible, while regions 94 and 92 are rigid.

As described with reference to FIGS. 1A-2C for device 20, mutatis mutandis, for some applications, device 60 comprises a release liner, typically configured such that each side region can be placed against a respective sidewall and each surface 22 can be placed in contact with a respective skin site 48, before release liner is pulled out from between mount 70 and the nose of the subject.

As described hereinabove for device 20, device 60 may be used for different applications (e.g., to treat different conditions), such as to treat dry eye, to increase efficacy of eye drops, to improve sleep, and/or to treat pain.

Nose pad 72 is at least partly shapeable to a contour of the nose of the subject, e.g., so as to facilitate contact, and thereby adhesion, between the skin of the nose and adhesive layer 26. Typically, nose pad 32 is shapeable, and adhesive layer 26 is disposed thereon, such that the adhesive layer is adherable to at least one sidewall 47 (e.g., both sidewalls) of the nose of the subject.

Typically, device 60 is left in place for a duration of at least 15 min, such as at least 1 hour. For some applications, e.g., for treating dry eye, device 60 is left in place for a duration of at least 5 hours (e.g., at least 8 hours and/or overnight while the subject sleeps), such that the device exerts a generally constant pressure on skin sites 48 for the duration.

As described for cushions 28, cushions 68 are typically elastically-compressible. For some applications, mount 70, longitudinal members 64, and/or longitudinal members 65 are rigid and/or malleable, and cushions 68 comprise and/or function as the sole elastically-deformable members. For some applications, side regions 91 are rigid, but region 90 is flexible, such that device 90 is bendable (e.g., foldable) over the nose of the subject. For some such applications, device 60 does not comprise longitudinal member 64 or longitudinal member 65.

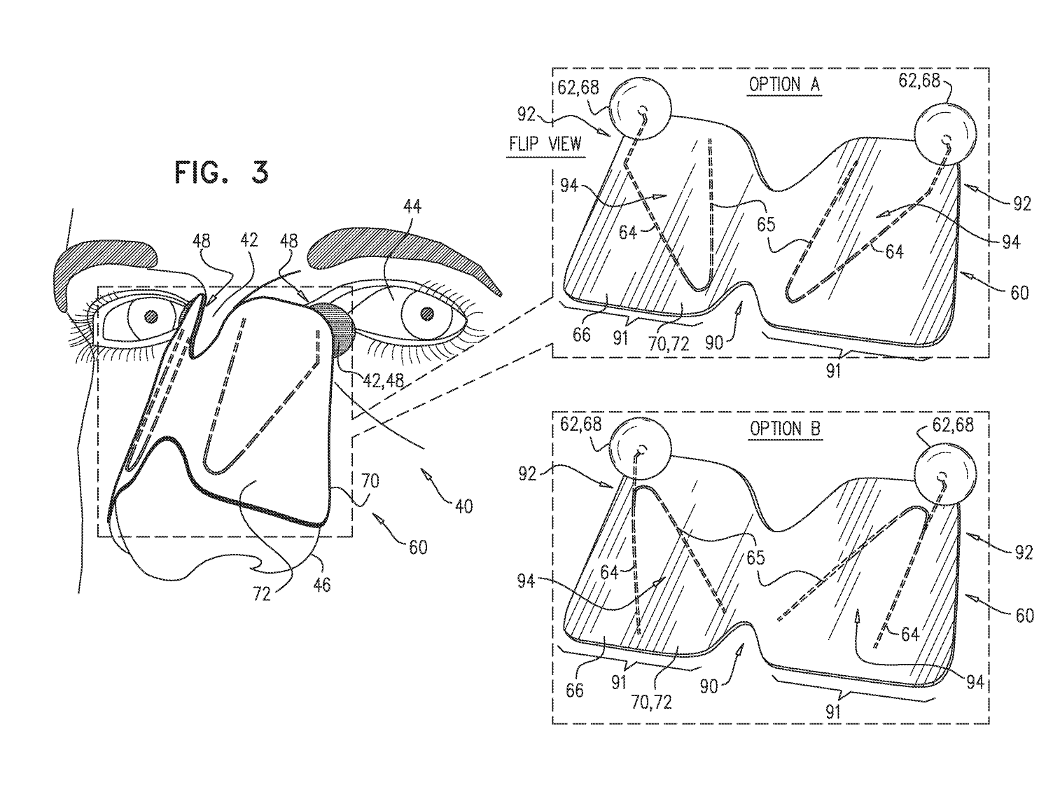

Reference is made to FIG. 4, which is a schematic illustration of a device 100 for treating a subject, in accordance with some applications of the invention. Device 100 has a plurality of skin-contacting surfaces 102, and comprises at least one elastically-deformable member, such as a longitudinal member 104, and an adhesive layer 106 (indicated in FIG. 4 as being on the reverse side of device 100). Typically, device 100 comprises a plurality of cushions 108 that define skin-contacting surfaces 102. Typically, device 100 comprises a mount 110, coupled to longitudinal member 104, and adhesive layer 106 is disposed on the mount. Further typically, device 100 comprises two longitudinal members 104, each longitudinal member being coupled to a respective cushion 108. Still further typically, mount 110 comprises a face pad, such as a nose pad 112, on which adhesive layer 106 is disposed. For example, a portion of mount 110 may serve as the face pad.

For some applications, components of device 100 are identical to, and/or function in the same manner as, identically-named components of device 20 and/or device 60, described hereinabove, mutatis mutandis, except as described.

Mount 110 is configured (e.g., shaped) to be adhered by adhesive layer 106 to nose 46 of the subject. Mount 110 typically defines a central core region 130 of device 100, and cushions 108 typically define respective extremity regions 132 of the device.

Typically, mount 110 is at least partly rigid, and longitudinal members 104 extend away from the mount such that when the mount is adhered to the face (e.g., nose) of the subject, the longitudinal members provide a pressing force that presses surfaces 102 against skin sites 48. Longitudinal members 104 typically have a first state in which surfaces 102 are closer together, and placement of the longitudinal members against skin sites 48 provides a deforming force that deforms the longitudinal members into a second state in which the surfaces are further apart (e.g., as described for device 20, mutatis mutandis). Longitudinal members 104 are configured to elastically return toward the first state, and thereby provide the pressing force.

As described hereinabove for devices 20 and 60, device 100 may be used for different applications (e.g., to treat different conditions), such as to treat dry eye, to increase efficacy of eye drops, to improve sleep, and/or to treat pain.

Typically, device 100 is left in place for a duration of at least 15 min, such as at least 1 hour. For some applications, e.g., for treating dry eye, device 100 is left in place for a duration of at least 5 hours (e.g., at least 8 hours and/or overnight while the subject sleeps), such that the device exerts a generally constant pressure on skin sites 48 for the duration.

As described for cushions 28, cushions 108 are typically elastically-compressible. For some applications, mount 110 and/or longitudinal members 104 are rigid and/or malleable, and cushions 108 comprise and/or function as the sole elastically-deformable members.

Reference is made to FIGS. 5A-C, which are schematic illustrations of adjustment mechanisms for adjusting a juxtaposition of (e.g., a distance between) adhesive layer 26 and/or central core portion 50 and surfaces 22 of device 20, in accordance with some applications of the invention. Each of the adjustment mechanisms is disposed (e.g., mechanically disposed) between (1) a respective surface 22, and (2) adhesive layer 26 and/or central core portion 50. Although the adjustment mechanisms are described as components of device 20, it is to be noted that devices 60 and 100 may also comprise such adjustment mechanisms, mutatis mutandis.

When device 20 is placed on and adhered to the face of the subject, the skin of the subject typically moves in response to forces applied by the device. Such movement may result in displacement of surfaces 22, a reduction in the pressing force applied by the surfaces to skin sites 48, and/or a change in direction in which the pressing force is applied to the skin sites. The adjustment mechanisms described with reference to FIGS. 5A-C are typically used to compensate for these effects. Alternatively or additionally, the adjustment mechanisms described with reference to FIGS. 5A-C may be used to compensate for asymmetry of the face of the subject or asymmetrical positioning of the device on the face of the subject.

FIG. 5A shows an adjustment mechanism 150, comprising a spring 152. Spring 152 may comprise a compression spring and/or a tension spring, and is typically configured to automatically adjust the distance between central core portion 50 and surfaces 22 of device 20. For example, in response to a reduced pressing force applied by surfaces 22 to skin sites 48, spring 152 may expand. For some applications, the user applies a force to spring 152 prior to, during, and/or after adherence of device 20 to the face, and subsequently releases the force.

For some applications, spring 152 is continuous with longitudinal member 24 (e.g., a single wire or strip is configured to serve as both longitudinal member 24 and spring 152).

FIG. 5B shows an adjustment mechanism 160, comprising a ratchet 162. Ratchet 162 is manually adjustable by a user (e.g., the subject being treated). Typically, ratchet 162 is adjusted subsequently to placement of surfaces 22 against skin sites 48 and adhesion of adhesive layer 26 to the face of the subject.

For some applications, ratchet 162 is configured to automatically adjust in response to an undesirably great pressing force. For example, ratchet 162 may be configured to alter a dimension of the device (e.g., a juxtaposition of adhesive layer 26 and surfaces 22, and/or a length of longitudinal member 24) in response to a pressing force that is greater than a threshold force (e.g., greater than 25 g force, e.g., greater than 50 g force, e.g., greater than 100 g force).

FIG. 5C shows an application in which device 20 comprises adjustment mechanism 150 and adjustment mechanism 160.

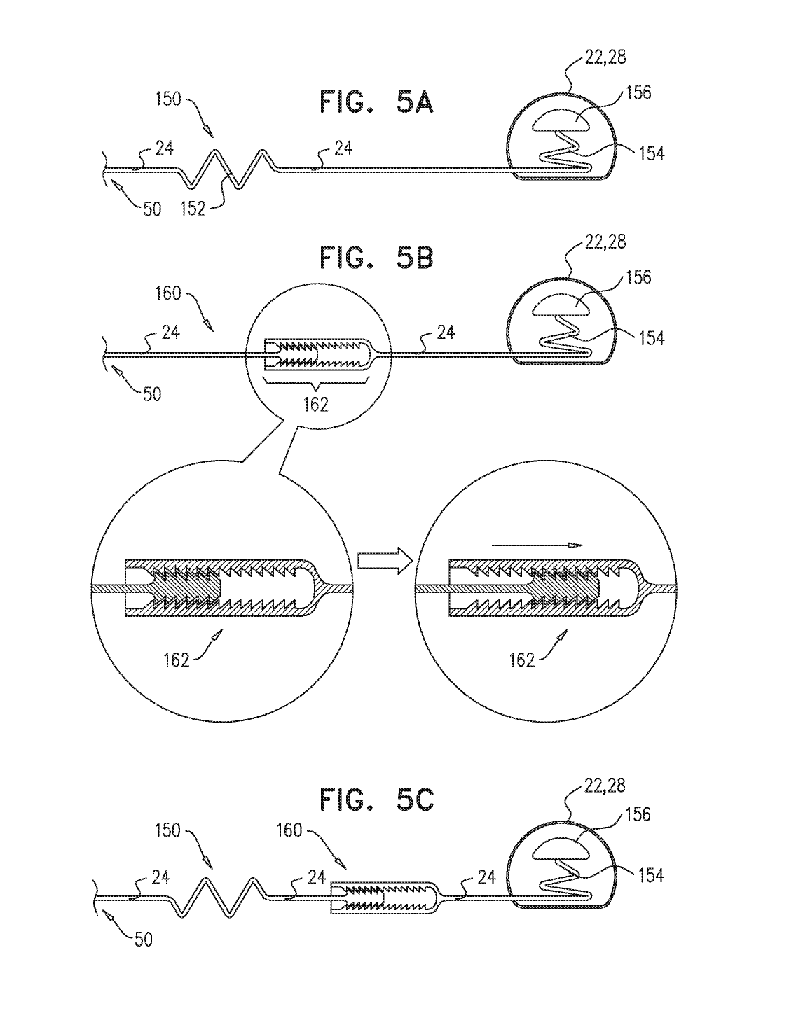

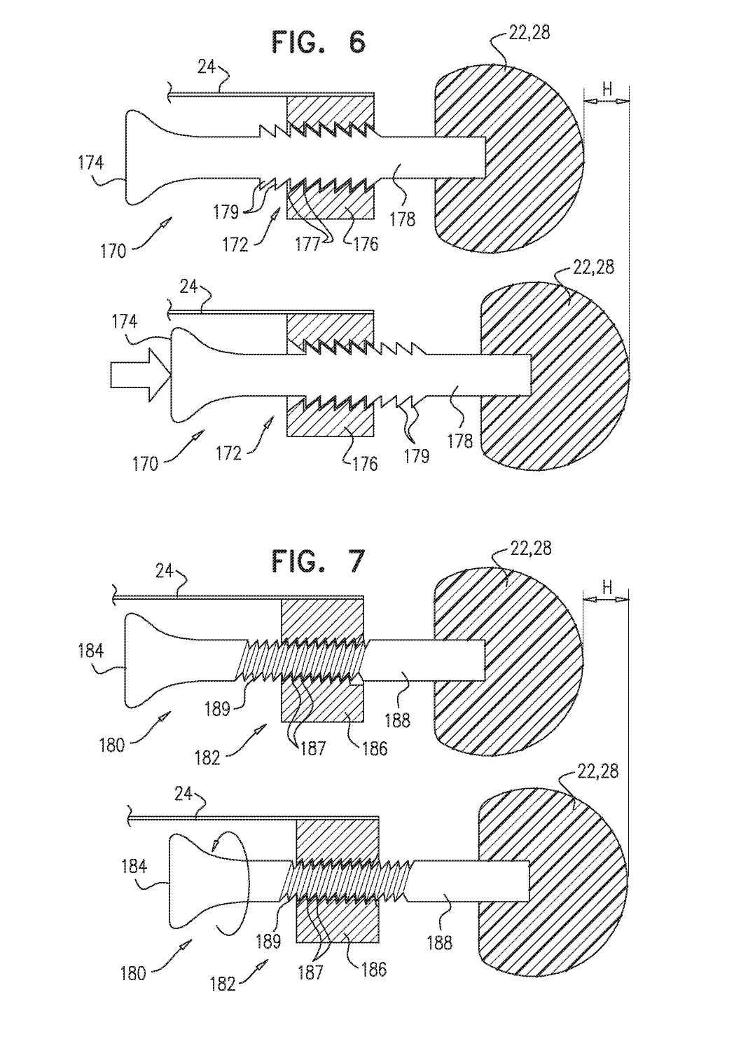

Reference is made to FIGS. 6-7, which are schematic illustrations of adjustment mechanisms for adjusting a juxtaposition of (e.g., a distance between) (1) skin-contacting surface 22, and (2) adhesive layer 26, longitudinal member 24 and/or mount 30, in accordance with some applications of the invention. Each of the adjustment mechanisms is disposed (e.g., mechanically disposed) between (1) a respective surface 22, and (2) adhesive layer 26 and/or longitudinal member 24. Although the adjustment mechanisms are described as components of device 20, it is to be noted that devices 60 and 100 may also comprise such adjustment mechanisms, mutatis mutandis.

As described hereinabove, when device 20 is placed on and adhered to the face of the subject, the skin of the subject typically moves in response to forces applied by the device, possibly resulting in displacement of surfaces 22 and/or a reduction in the pressing force applied by the surfaces to skin sites 48. Because of this effect, or independently of this effect, it may be desirable to adjust the force (e.g., a magnitude and/or a direction thereof) of the pressing force against skin sites 48. The adjustment mechanisms described with reference to FIGS. 6-7 are typically used to adjust the pressing force subsequently to adhering device 20 to the face of the subject. Alternatively or additionally, the adjustment mechanisms described with reference to FIGS. 5A-C may be used to compensate for asymmetry of the face of the subject or asymmetrical positioning of the device on the face of the subject.

FIG. 6 shows an adjustment mechanism 170, comprising a ratchet 172, in accordance with some applications of the invention. Ratchet 172 comprises a shaft 178 and a housing 176, coupled to longitudinal member 24 (and/or to mount 30; not shown in FIG. 6). Shaft 178 and housing 176 define ratcheting surfaces, such as teeth 179 and recesses 177. Although shaft 178 is shown as defining teeth 179, and housing 176 is shown as defining recesses 177, it is to be understood that the inverse configuration is also possible.

Using an adjuster 174 (e.g., a button surface), the user pushes shaft 178 through housing 176, such that surface 22 moves further away from longitudinal member 24. Typically, such movement increases the pressing force of surface 22 against skin site 48.

For some applications, ratchet 172 is configured to automatically adjust in response to an undesirably great pressing force. For example, ratchet 172 may be configured to alter a dimension of the device (e.g., a juxtaposition of (1) surface 22, and (2) adhesive 26 and/or longitudinal member 24) in response to a pressing force that is greater than a threshold force (e.g., greater than 25 g force, e.g., greater than 50 g force, e.g., greater than 100 g force).

FIG. 7 shows an adjustment mechanism 180, comprising a screw mechanism 182, in accordance with some applications of the invention. Screw mechanism 182 comprises a shaft 188 and a housing 186, coupled to longitudinal member 24 (and/or to mount 30; not shown in FIG. 7). Shaft 188 defines a first screw surface, such as an external screw thread 189, and housing 186 defines a second screw surface, such as an internal thread 187.

Using an adjuster 184, the user moves shaft 178 through housing 176 by rotating the shaft, such that surface 22 moves further away from, or closer to, longitudinal member 24. Typically, such movement increases or decreases the pressing force of surface 22 against skin site 48. For some applications, adjuster 184 comprises a knob, for rotating by hand. For some applications, adjuster 184 comprises a screw head, for rotating with a screwdriver.

Reference is again made to FIGS. 5A-7. It is to be understood that the adjustment mechanisms described may be positioned on and/or between various portions of device 20, and configured to adjust (e.g., automatically or manually) various dimensions of the device and/or forces applied by the device.

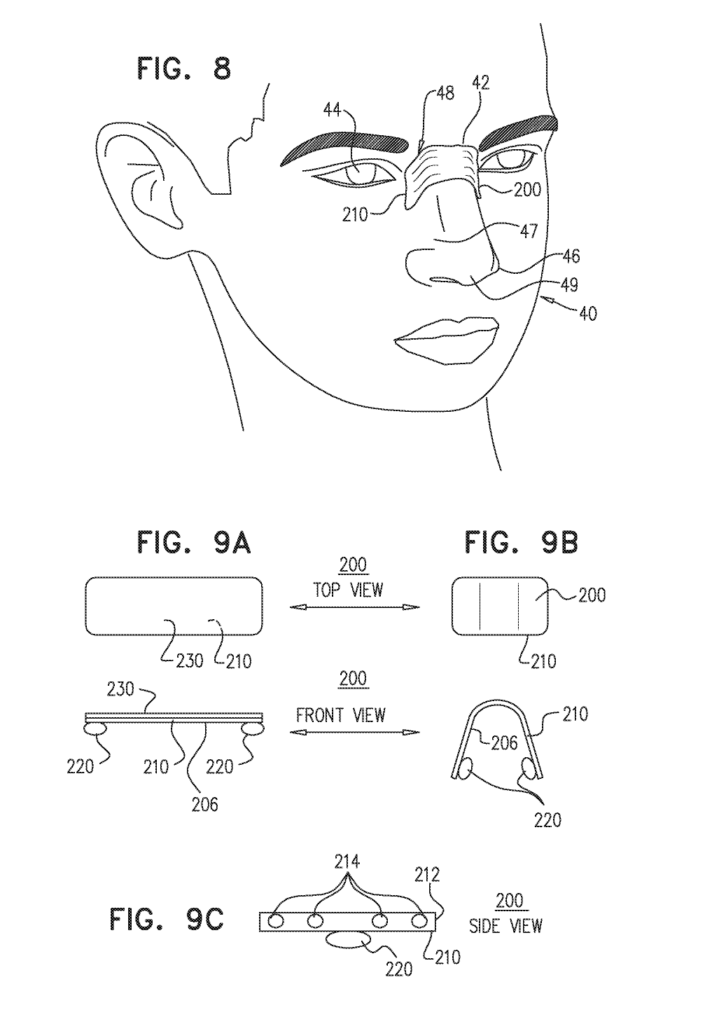

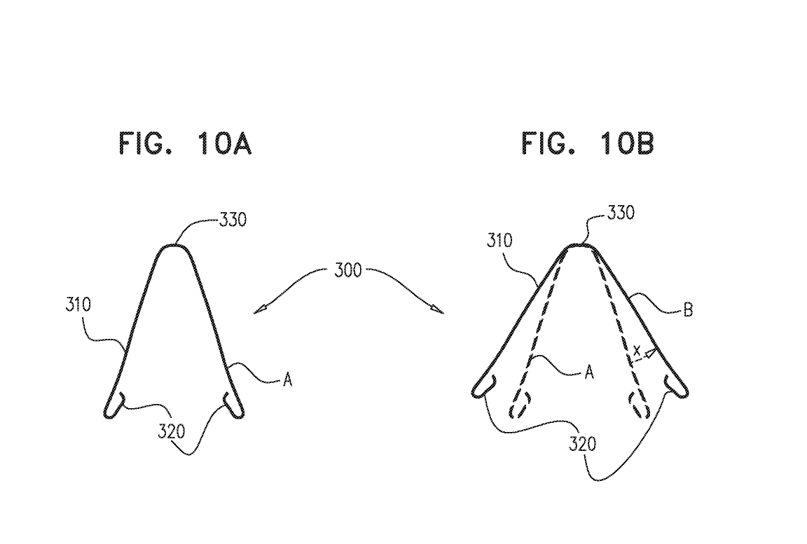

Reference is now made to FIGS. 8, 9A-C, and 10A-B, which are schematic illustrations of lacrimal drainage occlusion devices (e.g., canalicular occlusion devices, punctual occlusion devices and/or nasal strips) for treating a subject, in accordance with some applications of the invention.

The following preferred embodiments may be described in the context of exemplary eye dryness treating devices for ease of description and understanding.

However, the invention is not limited to the specifically described devices and methods, and may be adapted to various clinical applications without departing from the overall scope of the invention (e.g., as described elsewhere herein with respect to improvement of sleep, treatment of pain, and/or increasing efficacy of eye drops).

The present invention, in some embodiments thereof, relates to non-invasive devices and methods for treating or avoiding dry eyes, and in particular, to non-invasive lacrimal drainage occlusion devices and methods of use thereof. In preferred embodiments, the lacrimal drainage occlusion device is formed of a single-use disposable strip, made from low cost materials and applicable for mass production. In such embodiments, a wearer can replace the strips in a daily basis or as otherwise chosen, while preferably maintaining continuous treatment or prevention of excessive tear drainage.

In an aspect of some embodiments, there is provided a nasal strip comprising a malleable strip portion (e.g., a mount) sized and shaped to selectively surround, at least partly, a specific human nose portion. The human nose portion may include a nasal bridge, a lacrimal sac, and/or lacrimal canaliculi, or an adjacent portion therebelow or thereabove. The strip portion can have a specific shape and/or curvature such that it will efficiently fit and nest over the human nose portion without bulging to other nose portions or irritate the wearer.

The nasal strip may include at least one protruding portion outwardly protruding away from an inner side thereof, configured to press onto a skin site that is superficial to the lacrimal canaliculus (or other portion of the lacrimal drainage system, such as a lacrimal sac) when the nasal strip is deployed on said human nose portion. The protruding portion thereby defines a skin-contacting surface. The at least one protruding portion may be an integral part of the strip portion or a separate member connected thereto; and/or it may include a cushion-like body configured to maintain a chosen pressure or pressure range, when pressed to the lacrimal canaliculus at different allowed compression forces applied thereupon. The optional cushion-like body may include a viscoelastic material, be filled with compressible material or be partially filled with compressible or non-compressible material.

In some embodiments, the nasal strip self-compresses to a closed shape and is configured to clamp over the human nose portion, when deployed and left in place, therefore pressing the lacrimal canaliculus. In some such embodiments, the nasal strip includes at least one member (e.g., an elastically-deformable member; typically a longitudinal member) fixedly extending along a length of the strip portion, configured to compress the strip portion such that two end portions thereof meet at a nominal distance substantially smaller than a width of said human nose portion. The at least one member may include at least one elastic metal member (e.g., a wire or strip) bendable to a singular non-stressed shape.

In some embodiments, the nasal strip includes an adhesive layer provided on its inner side, configured to adhere the strip portion to the human nose portion and/or to the area over the lacrimal ducts (i.e., canaliculi) or sac. The adhesive layer may include bodily tissue acceptable adhesives, preferably temporary adhesives or such having light-to-moderate bonding force to the tissue, so that the wearer can remove the nasal strip without substantial pain or harm to the nose tissues and skin. In some embodiments, a disposable covering (e.g., a release liner) is provided over the adhesive layer and is removable prior to or after nasal strip deployment.

In some embodiments, the nasal strip includes a removable strip straightening means (e.g., a restraining member), configured to maintain said strip portion straight, and being removal prior or during nasal strip deployment, thereby allowing self-compression thereof. The straightening means may be an elastic, a rigid or a semi-rigid member or covering, removably provided attached, optionally to the external side of the nasal strip.

Referring back to the drawings, FIG. 8 schematically illustrates a deployed exemplary compression nasal strip 200. Nasal strip 200 is sized and curved such to fit in or adjacent the nasal bridge 42, as shown. Nasal strip 200 may be malleable therefore formable to nose structure. Nasal strip 200 may be elastic and/or stretchable to different lengths.

FIGS. 9A-C schematically illustrate different views and deployment modes of nasal strip 200. FIG. 9A shows a top view and a front view of nasal strip 200 in a straight mode (e.g., a first state), prior to deployment, whereas FIG. 9B shows same views of strip 200, now in bent or compressed mode (e.g., a second state). Nasal strip 200 includes a main body comprising a malleable strip portion 210 (e.g., a mount) covered with an adhesive layer 206 on its inner side. Optionally, and as shown in FIG. 9A, nasal strip 200 is provided with a removable straightening cover 230 (e.g., a restraining member) keeping it straighten until removal. Once optionally straightening cover 230 is removed, as shown in FIG. 9B, nasal strip 200 self-compresses to its bent form. Optionally, nasal strip 200 also includes at least one, now shown exemplary two, protruding members 220 (e.g., cushions) adapted to focus the force and pressurize more efficiently and effectively the lacrimal sacs and/or canaliculi. Such protruding members 220 may be configured to gently press the lacrimal sacs and/or canaliculi and or maintain substantial constant or shallow gradient pressure at different forces applied thereto by the nasal strip 200.

FIG. 9C shows a side cross-sectional view of a portion of nasal strip 200 after removal of any straightening cover. As shown, strip portion (e.g., mount) 210 includes a malleable (e.g., flexible) matrix 212, optionally synthetic, optionally textile, optionally polymeric; and a plurality of elastic metal wires 214 (e.g., elastically-deformable longitudinal members) embedded therein, and extending across the nasal strip length. Wires 214 provide skeletal strength to nasal strip 200 and stress it to shift its bent form.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Reference is now made to FIGS. 10A-B which schematically illustrate a different form of a malleable nasal strip shown as an exemplary compression wire 300. FIG. 10A shows compression wire 300 in nominal non-stressed form A (e.g., a first state), and FIG. 10B shows compression wire 300 stretched out to a stressed form B (e.g., a second state). Compression wire 300 includes an elastic wire 310 (e.g., an elastically-deformable longitudinal member) ending at both free ends 320 thereof. Optionally, each of the free ends 320 includes a form inwardly projected away from elastic wire 310 axis. In some embodiments, free ends 320 are twisted to a triangle-like or other form (for example, other polygon, curved shape or other) having at least one side and/or corner projecting inwardly, typically defining a skin-contacting surface and/or a cushion. When in non-stressed form A, compression wire 300 is in a predetermined bent or curved shape having an apex 330 enclosing a chosen angle.

In some embodiments, compression wire 300 is designed and configured such, that upon wearing onto a human nose portion including lacrimal canaliculi and/or a lacrimal sac, the compression wire 300 shall outwardly stretch up to allowed shift x, as shown in FIG. 10B. While stretching, compression wire 300 is stressed and tends to recover into nominal form A. In some embodiments, if free ends 320 are twisted as shown in the figures, the developed stress projects focused pressing forces to nose tissue (e.g., skin sites thereof) at the inwardly projected apexes, corners and/or sides. In some embodiments, compression wire 300 is designed such that these inwardly projecting apexes, corners and/or sides are meant to press lacrimal canaliculi and/or lacrimal sacs. Optionally, free ends 320 are adjustable to fine tune to direct contact with lacrimal canaliculi and/or lacrimal sacs at proper wearing.

In some embodiments, compression wire 300 or at least elastic wire 310 thereof is designed such to produce a minimal or mild pressure to lacrimal canaliculi and/or lacrimal sacs needed for partial or complete occlusion, yet avoiding harm when pressing for prolonged durations (e.g., greater than 5 minutes, optionally equal or greater than 15 minutes, optionally equal or greater than 30 minutes). In some such embodiments, compression wire 300 or at least elastic wire 310 are formed such that upon stretching to any position (e.g., according to shape and sized of covered nose portion), at least up to allowed shift x, the produced stress is substantially similar. Applicable material may be a superelastic material such as Ni—Ti alloy, optionally but not necessarily be designed such that when in contact with the nose portion, it will have a temperature slightly above its transformation temperature providing it in a martensitic form.

Optionally, alternatively or additionally, wire 300 includes at least two cushion elements (not shown) in the area of contact with the skin over the lacrimal canaliculi or sac for optionally avoiding or decreasing pain or discomfort. Optionally the cushions are disposable. In some embodiments, at least part of wire 300 (e.g., the cushions thereof, or apex 330) is at least partially covered with an adhesive layer, optionally for securing the placement of the compression device and prevent sliding or disconnection.

Reference is again made to FIGS. 8-10B. It is to be noted that, similarly to longitudinal members 24, 64, and 104 described hereinabove, elastic metal wires 214 and/or compression wire 300 may comprise nitinol, and may have some similar characteristics to these longitudinal members.

Reference is again made to FIGS. 1A-10B. Several components are described hereinabove as optionally comprising nitinol. Some advantages of using nitinol in applications described herein are described here:
Superelasticity of nitinol allows significant deformation of longitudinal members and springs described hereinabove. For example, longitudinal member 24 may be stretched to fit device 20 over a large range of nose bridge dimensions.
Similarly, but distinctly, nitinol provides (or can be configured to provide) relatively constant stress (e.g., spring force) at a range of strains (e.g., between 1 and 8 percent strain). This feature of nitinol allows a single device to be used on different subjects having a range of face dimensions (e.g., nose bridge dimensions), and to provide the same pressing force to the skin sites of those subjects.
For some applications, one or more nitinol components are configured such that the transformation temperature thereof is close to that of the skin of the subject (e.g., between 35 and 37 degrees C.). For example, spring 154 may be configured to alter a degree of compression thereof (e.g., to expand) in response to body heat of the subject (e.g., shortly after placement of device 20 on the face of the subject). Such a configuration may facilitate adhesion of the device to the nose of the subject while the pressing force is relatively low, and for the pressing force to increase subsequent to adhesion.

Reference is again made to FIGS. 1A-10B. It is hypothesized that the devices described herein may alternatively or additionally be used to facilitate relaxation of a subject, and thereby to treat conditions that benefit from relaxation, such as hypertension, heart failure, attention deficit hyperactivity disorder (ADHD), diabetes, stroke, and anxiety.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-127. (canceled)

128. Apparatus comprising:
a mount configured to be placed on a portion of a face of a subject;
at least one skin-contacting surface, said mount capable of providing a pressing force on said at least one skin-contacting surface; and
an adjustment mechanism configured to adjust a juxtaposition of said at least one skin-contacting surface relative to said mount such that after placing said mount on the portion of the face, said at least one skin-contacting surface is positioned to press on an anatomical site of a medial palpebral ligament of the subject, between a nasal bridge of the subject and an eye of the subject.

129. The apparatus according to claim 128, wherein said at least one skin-contacting surface comprises left and right skin-contacting surface positioned left and right relative to said mount.

130. The apparatus according to claim 128, wherein said at least one skin-contacting surface comprises a cushion.

131. The apparatus according to claim 128, wherein said mount comprises an elastically-deformable member capable of providing the pressing force.

132. The apparatus according to claim 131, wherein said elastically-deformable member is disposed between said at least one skin-contacting surface and said mount.

133. The apparatus according to claim 128, comprising an adhesive layer, disposed on the mount and not disposed on the at least one skin-contacting surface, and configured to adhere the mount to the portion of the face of the subject.

134. The apparatus according to claim 131, wherein said mount is configured to provide the pressing force across a range of strain values of the elastically-deformable member.

135. The apparatus according to claim 128, wherein said adjustment mechanism is configured to automatically adjust the juxtaposition of said at least one skin-contacting surface and said mount if a pressing force of said at least one skin-contacting surface exceeds a threshold force.

136. The apparatus according to claim 128, wherein said adjustment mechanism comprises a screw or a ratchet.

137. A method comprising:
placing the apparatus of claim 1 on the face of the subject, and using the adjustment mechanism to adjust the juxtaposition of said at least one skin-contacting surface relative to said mount such that after placing said mount on the portion of the face, said at least one skin-contacting surface applies a pressing force on the anatomical site of the medial palpebral ligament of the subject.

138. The method according to claim 137, wherein said pressing force on the anatomical site of the medial palpebral ligament is used to treat a condition of the subject.

139. The method according to claim 137, wherein said pressing force on the anatomical site of the medial palpebral ligament is used to facilitate relaxation of the subject.

140. The method according to claim 138, wherein the condition includes hypertension, heart failure, attention deficit hyperactivity disorder (ADHD), diabetes, stroke, migraine or anxiety.

141. The method according to claim 137, wherein said pressing force on the anatomical site of the medial palpebral ligament is used to improve sleep of the subject who suffers from suboptimal sleep.
